(19) United States
(12) Patent Application Publication
(10) Pub. No.: US 2007/0032460 A1
(43) Pub. Date: Feb. 8, 2007

(54) Use of voacamine and related compounds in the treatment of malaria

(76) Inventors: Leonard Stella, Pointe-Claire (CA); Soriba Cisse, Montreal (CA)

Correspondence Address: GEORGE A. SEABY;SEABY & ASSOCIATES, 250 CITY CENTRE AVNUE, OTTAWA, ON, K1R6K7, CA

(21) Appl. No.: 11/196,765

(22) Filed: Aug. 4, 2005

(51) Int. Cl. A61K 31/65; A61K 31/55; A61K 31/4706; A61K 31/53

(52) U.S. Cl. 514/154; 514/313; 514/452; 514/305; 514/241; 514/214.03

(57) ABSTRACT

Voacamine, voacamine isomers, metabolites and derivatives, and related compounds can be used to, in effect, reverse multi-drug resistance in malaria and are non-toxic. The compounds can be used in combination with known drugs such as chloroquine, arthemesin and qinghaosu to prevent or treat malaria.

### BACKGROUND OF THE INVENTION

This invention relates to compositions and methods for preventing and/or treating malaria.

In particular, the invention relates to the use of voacamine, voacamine isomers, metabolites, derivatives and related compounds for use in the prevention and/or treatment of malaria. The compounds are used alone or in combination with known antimalarial drugs to potentiate the effectiveness of such drugs against drug resistant malarial cells.

A number of different drugs have been found to be effective against malaria. However in many cases, the initial success of such drugs in the treatment and/or prevention of this disease is followed by total failure. Drugs that initially work become totally ineffective after a period of time. An initial period of remission is often followed by a period of frustration during which nothing seems to be effective against the disease. Death becomes inevitable. Such a phenomenon is commonly referred to as multi-drug resistance. A malarial cell that initially responds to treatment with one or more drugs becomes resistant to treatment by not only the drugs previously used, but also any other malarial treatment drugs.

Martin Odula and Milhous (Martin et al, Science, Feb. 28, 1987) disclosed the treatment of such multi-drug resistance in malaria by using verapamil. In “Reversal of Chloroquine Resistance in Plasmodium falciparum by Verapamil”, Martin et al., report that verapamil in combination with chloroquine was effective against malaria cells, but verapamil alone had no effect on malaria.

The problem with this approach is that verapamil is a calcium channel blocker. While calcium channel blockers are therapeutic in the treatment of hypertension at moderate levels, they are toxic at levels high enough for use with known anti-malarial drugs. Consequently, researchers throughout the world continue to press for techniques for, in effect, reversing multi-drug resistance. A successful clinical technique for reversing multi-drug resistance in malaria could be one of the most important breakthroughs in the fight against malaria.

### GENERAL DESCRIPTION OF THE INVENTION

The object of the present invention is to provide a treatment for use against certain multi-drug resistant parasitic diseases. In addition to having been observed in malaria, multi-drug resistance is a phenomenon, which has been observed in other parasitic diseases such as Entamoeba histolytica (amoebic dysentery), Trypanosoma (African sleeping sickness), Leishmania and AIDS pneumonia.

Accordingly, the invention relates to a method of preventing or treating malaria comprising the step of exposing malaria cells to an effective concentration of a compound of the formula 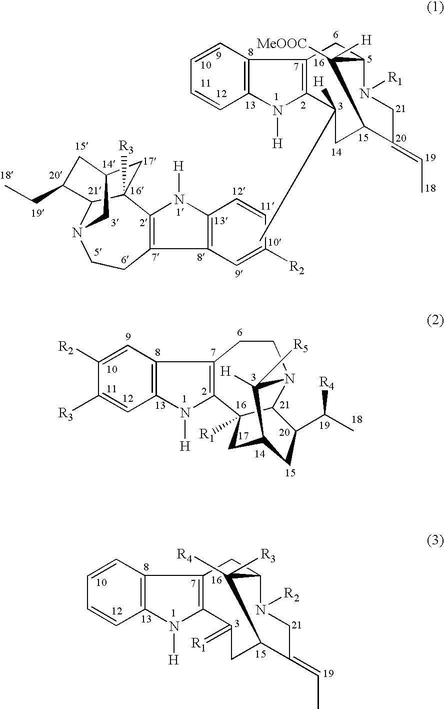
wherein R₁ is a methyl group or hydrogen, and R₂, R₃, R₄ and R₅, which are the same or different, are CH₂OH, CH₃, OCH₃, COOCH₃, OH or hydrogen in combination with at least one additional known principal drug used for preventing or treating malaria.

According to another aspect, the invention relates to a composition for a method of preventing or treating malaria comprising the compound of the formula 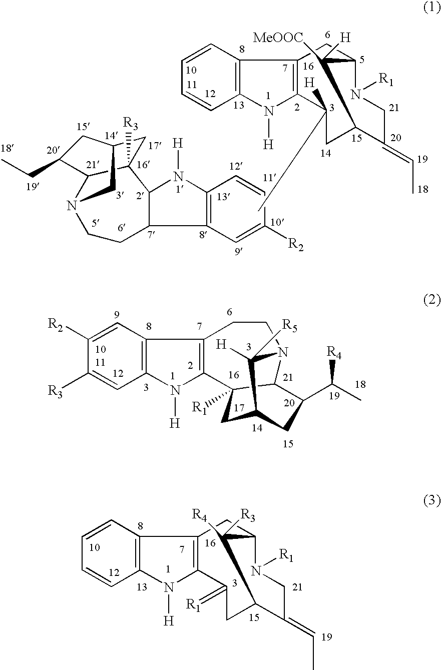
wherein R₁ is a methyl group or hydrogen, and R₂, R₃, R₄ and R₅, which are the same or different, are CH₂OH, CH₃, OCH₃, COOCH₃, OH or hydrogen in combination with at least one additional known principal drug used for preventing or treating malaria.

The inventor has determined that voacamine, voacamine isomers, metabolites and derivatives, and related compounds act to, in effect, reverse multi-drug resistance in malaria, and do not show any of the toxicity problems of verapamil.

Moreover, voacamine and the dimeric related compounds found in Peschiera laeta enhance vinblastine-mediated cytotbxicity in multi-drug resistant tumor cells (You M. et al., 1994, Journal of Natural Products). These dimeric alkaloids may also be effective at modulating the sensitivity of chloroquine resistant Plasmodium strains to this drug (Federici et al., 2000, Planta Medica).

Voacamine, voacamine, voacamine isomers, metabolites and derivatives, and related compounds are also specifically effective against malaria, including multi-drug resistant strains, even in the absence of primary treatment drugs. Voacamine, voacamine isomers, metabolites and derivatives are as effective against multi-drug resistant malarial strains as against drug sensitive strains.

### BRIEF DESCRIPTION OF THE DRAWINGS

FIG. 1 is an isobologram showing the effectiveness of voacamine and chloroquine at 50% inhibition concentrations against sensitive and resistant malarial strains; and

FIG. 2 is an isobologram showing the effectiveness of voacamine and quinghaosu at 50% inhibition concentrations against sensitive and resistant malarial strain.

### DETAILED DESCRIPTION OF THE INVENTION

In the preferred embodiment, the compounds of the present invention have the formula (1), (2) and (3) listed above. The compounds include voacamine, voacamine isomers and derivatives, and related compounds. In all of the examples, R₁ is a methyl group or hydrogen.

Variation within the group occurs in that R₂, R₃, R₄ and R₅ may be a methyl, methoxy, hydroxyl, carboxymethyl, or hydrogen and the isomeric configuration of the compounds at the C-1 position may be either R (rectus) or S (sinister). In addition, hernandezine includes a carboxymethyl group at the C-5 position, a substitution that does not appear to be significant in the operability of the compound. The specific manner in which the family members vary is set forth in Table V below, wherein the compounds are compared to two known drugs for activity against drug sensitive and drug resistant strains of P. falciparum malaria.

A specific in vivo dosage for each member of the voacamine family for reversing malarial multi-drug resistance and/or for specifically treating and/or preventing malaria has not been established. However, such dosage can be established through routine clinical experimentation by referencing the concentrations at which the various compounds have exhibited 50% inhibition as set forth in Tables I through V herein. These concentrations have been found to be from about 0.1 to about 3 micromolar. Such concentrations can be achieved in vivo by administering dosages of from about 100 to about 300 mg/day. It is known that at these concentrations, the voacamine family of compounds is substantially non-toxic. The preferred method for administering the drug is orally, although other methods such as injection maybe used.

One of the mechanisms whereby voacamine may sensitize chloroquine resistant strains may involve changes in rates of accumulation of chloroquine in the vacuole of chloroquine resistant parasites. The calcium channel blocker verapamil that has been known to reverse chloroquine resistance in strains such as K1 and W2 as a result of changes in membrane permeability exemplifies this phenomenon.

Prior studies of voacamine, voacamine isomers, metabolites, derivatives and related compounds for various other uses have indicated a minimal toxicity at doses of 2000 and 5000 mg/day. Voacamine and several voacamine derivatives were screened for calcium channel blocker activity, and such activity was found to be minimal. Thus, the toxicity problems associated with higher doses of calcium channel blockers such as verapamil have not so far been observed in members of the voacamine family.

The effectiveness of voacamine, voacamine isomers, metabolites, derivatives and related compounds in reversing malarial multi-drug resistance was determined by comparing the antimalarial action of voacamine, voacamine isomers, metabolites, derivatives and related compounds and chloroquine alone and in combination against a P. falciparum malarial strain that is sensitive to chloroquine and another strain resistant to chloroquine. A similar study was conducted using voacamine and qinghaosu. Chloroquine and qinghaosu are commonly used anti-malaria drugs.

The dose (IC₅₀) of each drug or each drug combination required to effect a 50% inhibition in the malarial activity of each strain was determined by establishing a dose response curve for each drug.

The non-resistant (D₆) strain and cloned Indochina (W₂) strain of P. falciparum were used. The former is sensitive to chloroquine and the latter is resistant to chloroquine. The two strains of the parasite were cultured according to the candle jar method of Trager and Jensen (Science, 1979, 193: 673-675). In a given experiment, 4-day-old Petri dish cultures (approx. 10% parasitemia) were diluted with medium containing an amount of non-infected type A human erythrocytes to obtain a culture with a final hematocrit of 1.5% and parasitemia of 0.5-10%. The resulting culture was ready for addition to microtitration plates with 96 flat-bottom wells.

The testing procedure used was similar to that described elsewhere (Desjardins et al., 1979, Antimicrobial Agents and Chemotherapy, 16: 710-718, 1979). Briefly, the final volume added to each of the 96-well microtitration plates was 250 μl and consisted of 25 μl of complete medium with or without the primary drug (chloroquine or qinghaosu), 175 μl of either the parasitized culture or a non-parasitized human erythrocyte control, and 25 μl of complete medium with or without voacamine, 25 μl radioactive 2,8-³H-hypoxanthine (0.5 μCi). The microtitration plates were incubated in a candle jar for an additional 18 hrs, at 37° C.

As the malaria parasite grows ³H-adenosine is metabolized and incorporates into polymeric RNA and DNA. The labeled polymers are trapped on glass fiber filters and unincorporated material is washed away. In the absence of drug there is 100% incorporation of the labeled material. When drugs interfere directly or indirectly, an inhibitory dose of 50% (IC₅₀) can be calculated (Van Dyke et al., 1987, Exp. Parasitol. vol. 64: 418-423).

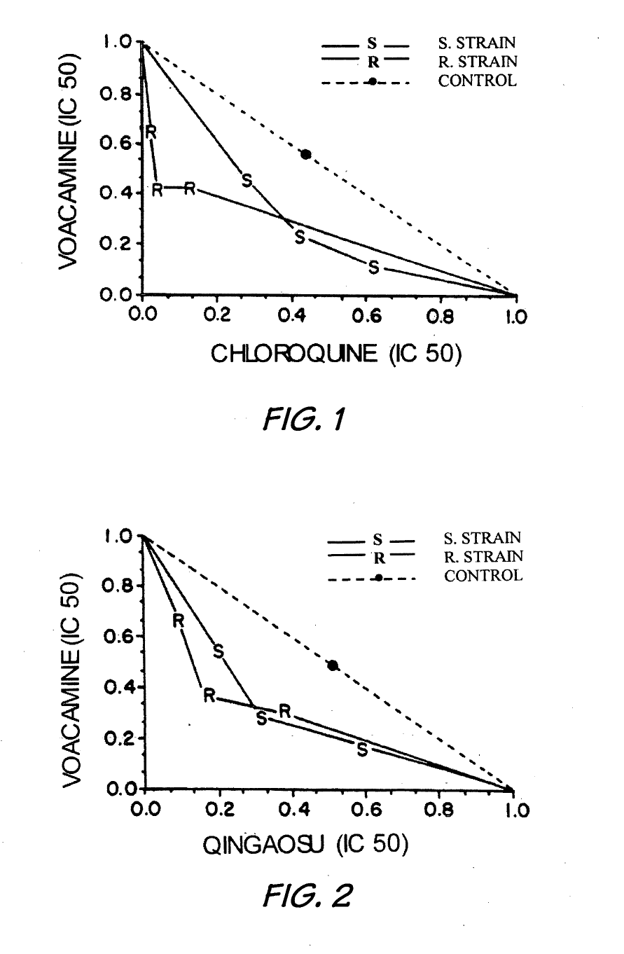

Voacamine ad the voacamine family of compounds completely reversed resistance to chloroquine in chloroquine-resistant malaria. When voacamine, voacamine isomers, metabolites, derivatives and related compounds are added to chloroquine, they supplement and potentiate the antimalarial activity. When voacamine is added to qinghaosu, it provides long-acting and synergistic activity to qinghaosu. This can be seen In Tables I, II, III, and IV while isobolograms (FIGS. 1 and 2) of voacamine and chloroquine as well as voacamine and qinghaosu reveal the synergistic and potentiating activity of voacamine when added to chloroquine or qinghaosu. Remarkably when 3.0 μMolar voacamine is added to 0.1 μMolar chloroquine, the IC₅₀ of chloroquine can be lowered 43-fold.

TABLE 1
IC₅₀ (nM) OF VOACAMINE (VOA) AND CQ FOR EACH DRUG ALONE AND IN COMBINATION*

| | | | DRUG COMBINATION** | | |
|---|---|---|---|---|---|
| | SINGLE DRUG | | VOA (1.0 μM) | VOA (2.0 μM) | VOA (3.0 μM) |
| MALARIA*** | VOA | CQ | CQ (0.3 μM) | CQ (0.2 μM) | CQ (0.1 μM) |
| S. STRAIN | 238.1 ± 23.7 | 28.5 ± 3.5 | 56.9 ± 8.2 (VOA) | 114.1 ± 25.1 (VOA) | 223.3 ± 35.8 (VOA) |
| | | | 15.9 ± 2.7 (CQ) | 13.5 ± 2.9 (CQ) | 8.2 ± 1.5 (CQ) |
| R. STRAIN | 290.5 ± 24.7 | 185.8 ± 4.9 | 79.5 ± 13.7 (VOA) | 125.5 ± 16.1 (VOA) | 254.6 ± 39.6 (VOA) |
| | | | 25.6 ± 3.2 (CQ) | 9.1 ± 2.1 (CQ) | 3.9 ± 0.5 (CQ) |

*The data in the table above are the mean values ± S.D. (nM) from three experiments except where noted
**Ratios of VOA/CQ in the drug combinations are 10:3, 10:1 and 30:1 respectively.
***S and R strains represent CQ-sensitive (D₆) and resistant (W₂) strains of P. falciparum respectively.

TABLE II
IC₅₀ (nM) OF VOA AND QHS FOR EACH DRUG ALONE AND IN COMBINATION*

| | | | DRUG COMBINATION** | | |
|---|---|---|---|---|---|
| | SINGLE DRUG | | VOA (1.0 μM) | VOA (2.0 μM) | VOA (3.0 μM) |
| MALARIA*** | VOA | QHS | QHS (0.3 μM) | QHS (0.2 μM) | QHS (0.1 μM) |
| S. STRAIN | 298.2 ± 59.8 | 38.3 ± 4.7 | 87.2 ± 9.5 (VOA) | 113.8 ± 5.6 (VOA) | 239.8 ± 45.3 (VOA) |
| | | | 25.9 ± 2.5 (QHS) | 14.6 ± 0.8 (QHS) | 9.7 ± 2.3 (QHS) |
| R. STRAIN | 305.1 ± 29.2 | 57.6 ± 4.5 | 78.9 ± 14.5 (VOA) | 98.1 ± 17.3 (VOA) | 296.9 ± 54.1 (VOA) |
| | | | 22.8 ± 3.1 (QHS) | 9.3 ± 1.5 (QHS) | 5.7 ± 1.2 (QHS) |

*The data in the table above are the mean values ± S.D. (nM) from three experiments except where noted
**Ratios of VOA/QHS in the drug combinations are 10:3, 10:1 and 30:1 respectively.
***S and R strains represent CQ-sensitive (D₆) and resistant (W₂) strains of P. falciparum respectively

TABLE III
EFFECT OF COMBINATION OF VOACAMINE
AND CHLORQUINE ON P. FALCIPARUM

| | | SFIC* | | |
|---|---|---|---|---|
| | | 1.0 μM (VOA) | 2.0 μM (VOA) | 3.0 μM (VOA) |
| MALARIA** | TRIAL | 0.3 μM (CQ) | 0.2 μM (CQ) | 0.1 μM (CQ) |
| S. STRAIN | 1 | 0.76 | 0.69 | 0.75 |
| | 2 | 0.65 | 0.76 | 0.69 |
| | 3 | 0.79 | 0.53 | 0.78 |
| | MEAN ± S.D. | 0.73 ± 0.04 | 0.66 ± 0.08 | 0.74 ± 0.03 |
| R. STRAIN | 1 | 0.62 | 0.54 | 0.70 |
| | 2 | 0.64 | 0.60 | 0.78 |
| | 3 | 0.43 | 0.29 | 0.54 |
| | MEAN ± S.D. | 0.56 ± 0.10 | 0.47 ± 0.12 | 0.71 ± 0.15 |

*SFIC represents sum of fractional inhibitory concentration as described by Berenbaum (11), SFIC is equal to one in cases of additive effects of the drugs, higher than one in cases of antagonism and lower than one in synergistic action.
**S and R strains: chloroquine sensitive (D₆) and resistant (W₂) strains of P. falciparum.

TABLE IV
EFFECT OF COMBINATION OF VOACAMINE
AND QINGHAOSU ON P. FALCIPARUM

| | | SFIC* | | |
|---|---|---|---|---|
| | | 1.0 μM (VOA) | 2.0 μM (VOA) | 3.0 μM (VOA) |
| MALARIA** | TRIAL | 0.3 μM (QHS) | 0.2 μM (QHS) | 0.1 μM (QHS) |
| S. STRAIN | 1 | 0.79 | 0.70 | 0.75 |
| | 2 | 0.71 | 0.44 | 0.78 |
| | 3 | 0.80 | 0.69 | 0.83 |
| | MEAN ± S.D. | 0.77 ± 0.08 | 0.61 ± 0.12 | 0.78 ± 0.05 |
| R STRAIN | 1 | 0.65 | 0.56 | 0.85 |
| | 2 | 0.79 | 0.68 | 0.67 |
| | 3 | 0.61 | 0.50 | 0.71 |
| | MEAN ± S.D. | 0.68 ± 0.07 | 0.58 ± 0.08 | 0.74 ± 0.14 |

*SFIC represents sum of fractional inhibitory concentration as described by Berenbaum (11), SFIC is equal to one in cases of additive effects of the drugs, higher than one in cases of antagonism and lower than one in synergistic action.

When the inhibiting activity of two drugs e.g. A and B are compared, the middle point of the dose response curve is usually chosen as the basis for comparison. This point is known as the inhibitory dose that occurs at the point of 50% inhibition of the response to be measured (inhibitory concentration at 50% inhibitory response=IC₅₀). An isobologram is developed by comparing the IC₅₀ of one drug against the other ( i.e. drug A against drug B). We start by putting the IC₅₀ of Drug B at the top of the Y-axis marked 1.0. The IC₅₀ of drug A is placed at the position 1.0 on the X-axis. Combinations of drug A and drug B are mixed and tested that are below IC₅₀ of either drug and the points are located on a graph. If the two drugs are additive there is a straight line between the Y₁X₀ (drug B) and Y₀X₁ (drug A). If the line or curve bends below the straight line the drugs are synergistic or potentiating. If the line bends above the straight line the two drugs are antagonistic (FIGS. 1 and 2).

Voacamine was also compared to several of its derivatives for their effectiveness against a chloroquine sensitive and a chloroquine resistant strain of P. falciparum malaria. The test procedure was basically the same as outlined above. The structural formulas of the derivatives are formulas (1), (2) and (3).

TABLE V
CHEMICAL STRUCTURE-ANTIMALARIAL ACTIVITY OF DIMERIC BISINDOLE
AND BASIC ALKALOIDS AGAINST PLASMODIUM FALCIPARUM IN VITRO

| | Substituents | | | | | | | | IC₅₀ (10⁻⁷ M) | | Ratio |
|---|---|---|---|---|---|---|---|---|---|---|---|
| ^{a}Drug | C3 | C4 | C10 | C10′ | C16 | C16′ | C19 | Linkage | S** | R** | (S/R)* |
| VOA | H | Me | — | OMe | CO₂Me | CO₂Me | — | C3-C11′ | 2.4 | 2.9 | 0.8 |
| DCV | H | Me | — | Me | CO₂Me | — | — | C3-C11′ | 4.9 | 8.2 | 0.6 |
| NDV | H | H | — | Me | CO₂Me | CO₂Me | — | C3-C11′ | 4.7 | 9.6 | 0.5 |
| VDN | H | Me | — | OMe | CO₂Me | CO₂Me | — | C3-C9′ | 5.1 | 9.8 | 0.5 |
| TAB | H | Me | — | OMe | CO₂Me | H | — | C3-C11′ | 6.5 | 9.5 | 0.7 |
| ERV | H | Me | — | OMe | CO₂Me | CO₂Me | — | C3-C11′ | 6.8 | 8.8 | 0.8 |
| VOB^{b} | ═O | Me | — | — | CO₂Me | — | — | — | 4.7 | 8.2 | 0.6 |
| COR^{b} | H | H | H | H | CO₂Me | — | H | — | 5.0 | 2.8 | 1.9 |

^{a}Dimeric Bisindole Alkaloids (Voa = Voacamine; DCV = Decarbomethoxyvoacamine; NDV = N₆Demethylvoacamine; VDN = Voacamidine; TAB = Tabernamine; ERV = Ervahanine)
^{b}Basic tertiary Alkaloids (VOB = Vobasine; COR = Coronaridine)
*IC₅₀ of a drug against sensitive strain of P. falciparum is divided by IC₅₀ for resistant strain.
**S and R represent chloroquine-sensitive and resistant strain of P. falciparum.

The results of Table V show that voacamine and its dimeric derivatives are far more effective against either the chloroquine sensitive malarial strain or the chloroquine resistant strain than the basic tertiary alkaloids. Coronaridine and vobasine were the best of the non-dimeric compounds, 2.8×10⁻⁷ and 8.2×10⁻⁷ moles were required respectively to effect a 50% inhibition in activity of the resistant strain, as compared to the IC₅₀ values from 13.3×10⁻⁷ up to 50.0×10⁻⁷ moles of the other non-dimeric voacamine family of compounds.

The results of Table V also illustrate the members of the voacamine family having at least one of the R₂ and R₃ substituents consisting of CH₃, OCH₃ or COOCH₃ that are the most effective against the chloroquine resistant malarial strains. When R₂ is a CH₃, OCH₃ or COOCH₃ substituent, the voacamine family members are actually as effective against the chloroquine resistant malarial strain as they are against the chloroquine sensitive malarial strain. This result suggests that the family members would also be the most effective members in affecting multi-drug resistance reversal. Thus, in the preferred voacamine family members at least one of R₂, R₃, R₄ and R₅ is CH₃, OCH₃ or COOCH₃, preferably at least R₂ being CH₃.

The results suggest that the compounds either inhibit the expression of the glycoprotein pump responsible for removal of the therapeutic drug from the cell or actually reverse or inhibit the pumping action of the glycoproteins or calcium channel associated with such multi-drug resistant cells. Instead of pumping all the toxic drug out of the cell, it appears that a lesser concentration of the toxic drug is being pumped out of the cell. At present, these are the only reasonable explanations for these surprising results, since the only known significant difference between the multi-drug resistant cells and the corresponding drug sensitive cells is the substantially greater percentage of P-glycoprotein associated with the multi-drug resistant cell.

1. A method of preventing or treating malaria comprising the step of exposing malaria cells to an effective concentration of a compound of the formula 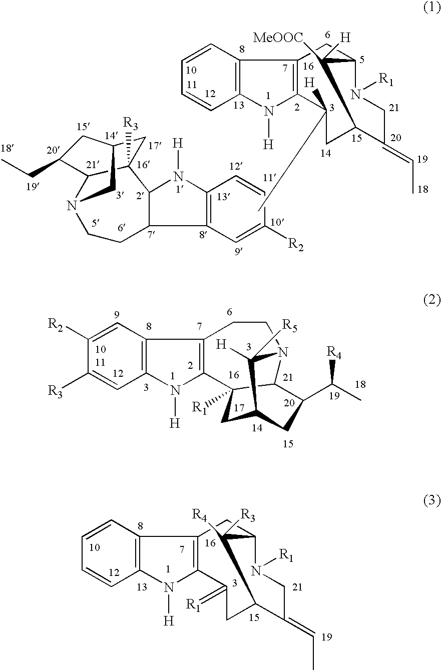
wherein R₁ is a methyl group or hydrogen and R₂, R₃, R₄ and R₅, which are the same or different, are CH₂OH, CH₃, OCH₃, COOCH₃, OH or hydrogen in combination with at least one additional known principal drug used for preventing or treating malaria.

2. The method of claim 1 wherein said principal drug is selected from the group consisting of chloroquine, arthemesin, qinghaosu and mixtures thereof.

3. The method of claim 1 wherein said compound is used at a dosage level of from about 100 to about 300 mg per day.

4. The method of claim 2, wherein said compound is used at a dosage level of from about 100 to about 300 mg per day.

5. The method of claim 1, wherein R₁ occupies location “S”.

6. The method of claim 1, wherein said compound is selected from the group consisting of voacamine, a voacamine isomer, a voacamine metabolite and a voacamine derivative.

7. The method of claim 6, wherein said compound is used at a dosage level of from about 100 to about 300 mg per day.

8. The method of claim 5, wherein said principal drug is selected from the group consisting of chloroquine, arthemesin, qinghaosu and mixtures thereof.

9. The method of claim 6, wherein said principal drug is selected from the group consisting of chloroquine, arthemesin, qinghaosu and mixtures thereof.

10. The method of claim 7, wherein said principal drug is selected from the group consisting of chloroquine, arthemesin, qinghaosu and mixtures thereof.

11. The method of claim 7, wherein said principal drug is selected from the group consisting of chloroquine, arthemesin, qinghaosu , 8-aminoquinoline, amodiaquine, arteether, artemether, artemsinin, artesunate, artesunic acid, artelinic acid, atovoquone, azithromycine, biguanide, chloroquine, chloroquine phosphate, chlorproguanil, cycloguanil, dapsone, desbutyl halofantrine, desipramine, doxycycline, dihydrofolate reductase inhibitors, dipyridamole, halofantrine, haloperidol, hydroxychloroquine sulfate, imipramine, mefloquine, penfluridol, phospholipid inhibitors, primaquine, proguanil, pyrimethamine, pyronaridine, quinine, quinidine, quinacrineartemisinin, sulfonamides, sulfones, sulfadoxine, sulfalene, tafenoquine, tetracycline, tetrandine, triazine or derivatives thereof.

12. A composition for preventing or treating malaria comprising a compound of the formula 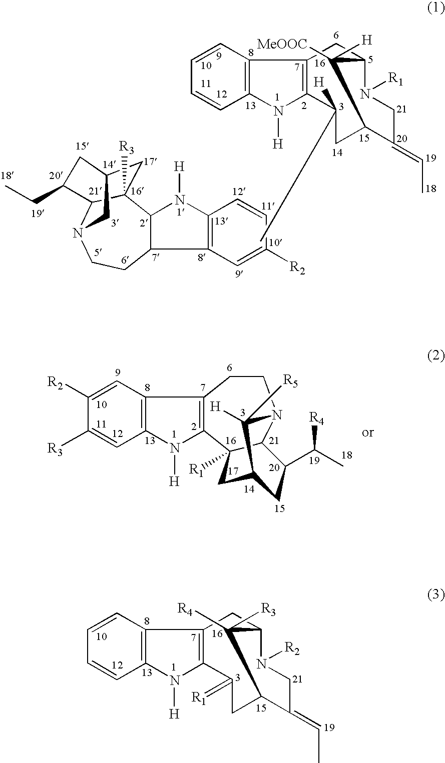
wherein R₁ is a methyl group or hydrogen and R₂, R₃, R₄ and R₅, which are the same or different, are CH₂OH, CH₃, OCH₃, COOCH₃, OH or hydrogen in combination with at least one additional known principal drug used for preventing or treating malaria.

13. The composition of claim 12, wherein said principal drug is selected from the group consisting of chloroquine, arthemesin, qinghaosu and mixtures thereof.

14. The composition of claim 12 wherein said compound is used at a dosage level of from about 100 to about 300 mg per day.

15. The composition of claim 13, wherein said compound is used at a dosage level of from about 100 to about 300 mg per day.

16. The composition of claim 12, wherein R₁ occupies location “S”.

17. The composition of claim 12, wherein said compound is selected from the group consisting of voacamine, a voacamine isomer, a voacamine metabolite and a voacamine derivative.

18. The composition of claim 17, wherein said compound is used at a dosage level of from about 100 to about 300 mg per day.

19. The composition of claim 15, wherein said principal drug is selected from the group consisting of chloroquine, arthemesin, qinghaosu and mixtures thereof.

20. The composition of claim 16, wherein said principal drug is selected from the group consisting of chloroquine, arthemesin, qinghaosu and mixtures thereof.

21. The composition of claim 17, wherein said principal drug is selected from the group consisting of chloroquine, arthemesin, qinghaosu and mixtures thereof.

22. The composition of claim 18, wherein said principal drug is selected from the group consisting of chloroquine, arthemesin, qinghaosu , 8-aminoquinoline, amodiaquine, arteether, artemether, artemsinin, artesunate, artesunic acid, artelinic acid, atovoquone, azithromycine, biguanide, chloroquine, chloroquine phosphate, chlorprogdanil, cycloguanil, dapsone, desbutyl halofantrine, desipramine, doxycycline, dihydrofolate reductase inhibitors, dipyridamole, halofantrine, haloperidol, hydroxychloroquine sulfate, imipramine, mefloquine, penfluridol, phospholipid inhibitors, primaquine, proguanil, pyrimethamine, pyronaridine, quinine, quinidine, quinacrineartemisinin, sulfonamides, sulfones, sulfadoxine, sulfalene, tafenoquine, tetracycline, tetrandine, triazine or derivatives thereof.
